SEQUENCE LISTING (6 sequences)

SEQ ID NO: 1 - DNA, 4612 bp, Homo sapiens
gcatggcctgccgggagggggcaggtagccggcgggcccggtccaatgggtgccggcttccgaggagagggcggaggagaggaggaaggaggcgaactgtgggccccggccccattcattgccgtggccggcgggcactggggccccgtgttttcagagtcatggaggcgctaattcctgtcataaacaagctccaggacgtcttcaacacggtgggcgccgacatcatccagctgcctcaaatcgtcgtagtgggaacgcagagcagcggaaagagctcagtgctagaaagcctggtggggagggacctgcttcccagaggtactggaattgtcacccggagacctctcattctgcaactggtccatgtttcacaagaagataaacggaaaacaacaggagaagaaaatggggtggaagcagaagaatggggtaaatttcttcacaccaaaaataagctttacacggattttgatgaaattcgacaagaaattgaaaatgaaacagaaagaatttcaggaaataataagggagtaagccctgaaccaattcatcttaagattttttcacccaacgttgtcaatttgacacttgtggatttgccaggaatgaccaaggtgcctgtaggtgatcaacctaaggatattgagcttcaaatcagagagctcattcttcggttcatcagtaatcctaattccattatcctcgctgtcactgctgctaatacagatatggcaacatcagaggcacttaaaatttcaagagaggtagatccagatggtcgcagaaccctagctgtaatcactaaacttgatctcatggatgcgggtactgatgccatggatgtattgatgggaagggttattccagtcaaacttggaataattggagtagttaacaggagccagctagatattaacaacaagaagagtgtaactgattcaatccgtgatgagtatgcttttcttcaaaagaaatatccatctctggccaatagaaatggaacaaagtatcttgctaggactctaaacaggttactgatgcatcacatcagagattgtttaccagagttgaaaacaagaataaatgttctagctgctcagtatcagtctcttctaaatagctacggtgaacccgtggatgataaaagtgctactttactccaacttattaccaaatttgccacagaatattgtaacactattgaaggaactgcaaaatatattgaaacttcggagctatgcggtggtgctagaatttgttatattttccatgagacttttgggcgaaccttagaatctgttgatccacttggtggccttaacactattgacattttgactgccattagaaatgctactggtcctcgtcctgctttatttgtgcctgaggtttcatttgagttactggtgaagcggcaaatcaaacgtctagaagagcccagcctccgctgtgtggaactggttcatgaggaaatgcaaaggatcattcagcactgtagcaattacagtacacaggaattgttacgatttcctaaacttcatgatgccatagttgaagtggtgacttgtcttcttcgtaaaaggttgcctgttacaaatgaaatggtccataacttagtggcaattgaactggcttatatcaacacaaaacatccagactttgctgatgcttgtgggctaatgaacaataatatagaggaacaaaggagaaacaggctagccagagaattaccttcagctgtatcacgagacaagtcttctaaagttccaagtgctttggcacctgcctcccaggagccctcccccgctgcttctgctgaggctgatggcaagttaattcaggacagcagaagagaaactaaaaatgttgcatctggaggtggtggggttggagatggtgttcaagaaccaaccacaggcaactggagaggaatgctgaaaacttcaaaagctgaagagttattagcagaagaaaaatcaaaacccattccaattatgccagccagtccacaaaaaggtcatgccgtgaacctgctagatgtgccagttcctgttgcacgaaaactatctgctcgggaacagcgagattgtgaggttattgaacgactcattaaatcatattttctcattgtcagaaagaatattcaagacagtgtgccaaaggcagtaatgcattttttggttaatcatgtgaaagacactcttcagagtgagctagtaggccagctgtataaatcatccttattggatgatcttctgacagaatctgaggacatggcacagcgcaggaaagaagcagctgatatgctaaaggcattacaaggagccagtcaaattattgctgaaatccgggagactcatctttggtgaagagaactatgtaatactgagactttgttgactcaaaacttgctagttactgcctacctgagtagaatcttatttatgaactcctgtgtattgcaatggtatgaatctgctcatgtggagactggctataaactgaaaagtgtattccaaattgcagaacacatcacacatttaatccaaataataaatggctgtttctaaagtttcccagtatatataaaatacatcaagtctgtcttgtgacagtttcatctgaacttaacttaaaaacaactgttaatgttctagttgtgcaaagcagtttgcctgtggataagatgacctgtgtaataatctttgttagtagtcttaaagctgctgccatagtcctccaagaagaaagcaccaagacaacatttcatatgactataatgcatgtactatataagctgatctggctttgaaagatgtgagttggcaagttcctcacatagagtcattgtattccacctgtccttcaatttagttttttctgagcttctttgcagcctttgatgtgtttttaagaaagctgaatgcacaagaggatctgtgacactgacatggctgtggtgtgcatactgtgtagttacatagcccttccaattctgggtccatttgcactagcaaattaaaatatgctttgattcatacttaaacctgaaagcaggaatgcctacattaattcctacattaaaaacagccatctacccttgattatctagaaagacttggtaatgatggtcagttccttttagatttcagaaaatcaaatgatgacctaaatttcccttaatttgcaaatacagtagtaattaaggtacatctctaaagtggagcacttacaccaggctctaagattcactttgaggtggaacttaaaaccagtgtactgtatgtatgcattggtaatagctacttttgcttcatagcttcataccaacaaaatatatttattagaatagtatgaaagtactggaggagctgaaagaaaaacacccaaggctgggcgtggtggcacacgcctgtaatcccagcactttgggaggccgaggcaggtggatcacctgaggttgggagttggagaccagcttgaccaacatggagaaaccccgtctctactaaaaatacaaaattggccgggcgtggtggcgcatgcctgtaatcccagctactcgggagggtgaggcaggagaattgcttgaccctgggaggtggaggttgtggtgagctaagatcgtgccattgcactccagccttggcaacaagagcgaaactccgtctcaaaaaaaaaaaataaaacaacacccagatagatacacatactccttcagacttacagacctaagctgcatttatggggtagtgatgaggtttagaacatatacatattttgttaaaattccccagatgattcttggtatgaacgactatattataaattttaagatgtacttagaaatccttaagacatctagccccgtctctaatagacaacacatttatattgcagatattacttttttttcagtttatgaccaggtatttatgaaggactattggcagggaaaatatgaatatgttaactttagcttatggcatcaatttactaaggaacaacaggctcaccaactgatgtcaaacataaaaacccccacatcagtctgatacgatatggtactactttgaatctgttactagtaccatcttgacagaggatacatgctcccaaaacgtttgttaccacacttaaaaatcactgccatcattaagcatcagtttcaaaattatagccattcatgatttactttttccagatgactatcattattctagtcctttgaatttgtaaggggaaaaaaaacaaaaacaaaaacttacgatgcacttttctccagcacatcagatttcaaattgaaaattaaagacatgctatggtaatgcacttgctagtactacacactttgtacaacaaaaaacagaggcaagaaacaacggaaagagaaaagccttcctttgttggcccttaaactgagtcaagatctgaaatgtagagatgatctctgacgatacctgtatgttcttattgtgtaaataaaattgctggtatgaaatgacactaaagtttgtcaaaaaatgaattcttaacttttctcccagagaaagggagacaaaaggagctttttaatacctaatctactttggaacataaccgtatagag

SEQ ID NO: 2 - protein, 736 aa, Homo sapiens
MEALIPVINKLQDVFNTVGADIIQLPQIVVVGTQSSGKSSVLESLVGRDLLPRGTGIVTRRPLILQLVHVSQEDKRKTTGEENGVEAEEWGKFLHTKNKLYTDFDEIRQEIENETERISGNNKGVSPEPIHLKIFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKSSKVPSALAPASQEPSPAASAEADGKLIQDSRRETKNVASGGGGVGDGVQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 3 - DNA, 2515 bp, rattus norvegicus
ggcgcgtggcctgccgggagcgggctgggaggtggcggcctagcccaatgggtgcgtgctcccgaggagagggcggaggagaaaaggaagcaagcgggctgcgggctgcgtccccattcattgcagtggtctgctgggttggggtccagtgtttccggagtcatggaggcgctgatcccggtcatcaataagctgcaagacgtcttcaacaccgtgggcgccgacatcatccagctgcctcagattgtcgtagtgggaactcagagcagtggaaagagctcagtgctggaaagcctagtggggagggaccttcttcccagagggactggtgtggtcacccggagacctcttattctgcagctggtccacgtttcaccagaagataaaagaaaaacaacaggagaagaaaatggagttgaagcagaagaatggggtaaatttcttcacaccaaaaacaagctttacacagattttgatgaaattcgacaagaaattgaaaatgaaactgaaagaatttcaggaaataataagggagtaagccctgagccaatccatctcaaggttttctcgcccaacgttgtcaacctgacgcttgtggatttaccaggaatgaccaaagtacctgtaggtgatcagcccaaggatattgagcttcaaatcagagaacttattcttcggttcatcagtaatcccaattccattatccttgctgtcactgctgcaaatacagatatggcaacgtcagaggcacttaagatttccagagaggtagatccagatgggcgcagaactctagctgtaatcaccaagcttgatctcatggatgcgggtactgatgccatggatgtattgatgggaagggttattccagtcaagcttggaataattggagtagttaacaggagccaactggacattaacaataagaagagtgtaactgattcaatccgtgatgagtatgcttttcttcaaaagaagtacccatctctggccaacagaaatggaacaaagtatcttgctaggactctgaataggttacttatgcatcatatcagagactgtttaccagagctgaaaacaagaataaatgtcttagctgctcagtatcagtctcttctaaatagctatggtgagcccgtggatgataaaagtgctactttactccagctcattaccaagtttgccacagagtattgtaacactattgaaggaacggcaaagtacattgaaacttctgagctatgtggtggtgctaggatttgttatattttccatgagacttttgggcgaaccttagaatctgttgatccactaggtggccttaacactattgacatcttgaccgccattagaaatgctactggcccccgtccagctttatttgtgcctgaagtttcatttgagttactggtcaaacgtcagattaagcgtctagaggagcccagcctccgctgtgtggagctggtccacgaggaaatgcagaggatcattcagcactgtagcaactacagcacacaggaactgttacggttccctaaactccatgatgccatagttgaagtagtgacttgtcttcttcgtaaaaggttgcccgtgacaaatgaaatggtacataacttagtggcaattgagctagcatatatcaacacaaaacaccccgactttgctgatgcctgtgggctaatgaacaataacatagaggaacaaagaagaaacagactagcaagagaactaccttccgctgtatcgcgagacaagttaattcaggacaacagaagagaaactaaaaatgttgcatctgcaggtggtggaattggagatggtggtcgaattggagatggtggtcaggaaccgacaacaggcaactggagaggaatgctgaaaacttcaaaagctgaagaattacttgctgaagaaaaatcaaaaccaattccaattatgccagcaagtccacaaaaaggccatgctgtcaatttgctagatgtgccagttccagttgcacgaaaactgtctgcccgagaacagcgagattgtgaggttattgaaagactcatcaaatcatattttctaattgtcagaaagaatattcaagacagcgtcccaaaggcagtaatgcattttttggttaatcatgtgaaagatactcttcagagtgaattagtagggcagctgtataagtcatccttattagatgaccttctgactgaatccgaggacatggcacagcgaagaaaagaagcggctgacatgctgaaggcattacaaggagccagtcaaattattgctgaaatccgagagactcatctttggtgaacagaactgtttggtcttctacactttgctgacttgaacctactagctactgcctacctgagtagaaatttatttatggacttttgtgtatcgcagtgtatgaatttgcttgtgtgaagactggctataagctgaaaagtatattattcattacaagacaaatcacatgtttaattcaaaaaaaaaaaaaaaaaaaaaaaaa

SEQ ID NO: 4 - protein, 716 aa, rattus norvegicus
MEALIPVINKLQDVFNTVGADIIQLPQIVVVGTQSSGKSSVLESLVGRDLLPRGTGVVTRRPLILQLVHVSPEDKRKTTGEENGVEAEEWGKFLHTKNKLYTDFDEIRQEIENETERISGNNKGVSPEPIHLKVFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAVSRDKLIQDNRRETKNVASAGGGIGDGGRIGDGGQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

SEQ ID NO: 5 - DNA, 4078 bp, Mus musculus
gtcgtggcgcgtggcctgccgggagcgggctgggtgctggcccggcccaatgggtgcgggctcccgaggaaagggcggaggagaagaggaagcaagcgggctgtgggtcgagtccccattcattgcagtggcctgctgggttgggggtccagtgtttcgggggtcatggaggcgctgatcccggtcatcaataagctgcaggacgtcttcaacacagtgggtgcggacatcatccagctgcctcagatcgtcgtagtgggaacgcagagcagtgggaagagctcagtgctggaaagcctagtgggcagggaccttcttcccagaggaactggtgtggtcacccggagacctctcattctgcagctagtccacgtttcaccagaagataaaagaaaaacaacaggagaagaaaatgaccctgccacatggaaaaactcaagacacctttccaaaggagttgaagcagaagaatggggtaaatttcttcacaccaaaaacaagctttacacagattttgatgaaattcgacaagaaattgaaaatgaaactgaaagaatttcaggaaataataagggggtaagccctgagccaatccatctcaaggttttctcgcccaacgttgtcaacctgacacttgtggatttaccgggaatgaccaaagtacctgtaggcgatcagcccaaggacatcgagcttcagatcagagaacttattcttcggttcatcagtaatcccaattccattatcctcgccgtcactgctgcaaatacagatatggcaacatcagaagcactcaagatttccagagaggtagatccagatgggcgcagaactctagctgtaatcactaaacttgatctcatggatgcgggtactgatgccatggatgtattgatgggaagggttattccagtcaagcttggaataattggagtagttaacagaagccaactggatattaacaataagaagagtgtaactgattcaatccgtgatgagtatgcttttcttcaaaagaagtacccatctctggccaacagaaatggaacaaagtatcttgctaggaccctgaataggttacttatgcatcatatcagagattgtttaccagagctgaaaacaagaataaatgtcttagctgctcagtatcagtctcttctaaatagctatggtgaaccggtggatgataaaagtgctactttactccagcttattaccaaatttgccacagagtattgtaacacgattgaaggaaccgcaaagtacattgaaacttctgagctatgcggtggtgctaggatttgttatattttccatgagactttcgggcgaaccttagaatctgtggacccactaggtggccttaacactattgacatcctgactgccatcagaaatgctactggcccccgtcctgctttatttgtgcctgaagtttcatttgagttactggtcaaacgtcagattaagcgtctagaagagcccagcctacggtgtgtggagctggtccatgaggagatgcagaggatcattcagcattgtagcaattacagcacacaggaattgttacggttccctaaacttcacgatgccatagttgaagtagtgacctgtcttcttcgtaaaaggttgcccgtgacaaatgaaatggtgcataacttagtggcaattgagctagcgtatatcaacacaaaacaccccgactttgctgatgcctgtgggctaatgaacaataatatagaggaacaaagaagaaacaggctagcaagagagctgccttcagctggatcacgggacaaggtcccatctgcaggtggtgggattggagacggtggtcaggaaccaacaacaggcaactggagaggaatgctgaaaacttcaaaagctgaagaattacttgctgaagaaaaatcaaaaccaattccaattatgccagcaagtccacagaaaggccatgctgtcaatttgctagatgtgccagttccagttgcaagaaaactgtctgcccgagaacagcgagattgtgaggttattgaaagacttatcaaatcatattttctaattgtcagaaagaatattcaagacagtgtcccaaaggcagtaatgcattttttggttaatcatgtgaaagatactcttcagagtgaactggtagggcagctgtataagtcatccttattagatgaccttctgactgaatccgaggacatggcccaacgaagaaaagaagcagcggatatgctgaaggcattacaaggagccagtcaaattattgctgaaatccgagagactcatctttggtgaacagaactgtttgatatccacactttgctgacttgaacctgctgctgcctacctgagtagaagtttatgaacttttgtgtattgcagtgtatgaatttgctcgtgtgaagactggctataagctgaaaagtatattattcgttacaaaacaaatcacatgtttaattcacatttttcaagtttcctatgacgtatatcaagtctgtccgtttcagtcgaacttacaagagcaattgatattctagttgtacaaagcagtttgcctgtgcatatgacccatgtaactttcttagtctcaaagctgcagctgtagtcctcagaaaggaagctccaggacatttcatgactataatgcatgcactatttatatagactgacctggctttaaaagatggagcctggcaagttcctcacataatcaatgtatccacctatccttcatttttatagtcttaaaaaacaaacaaaactcttttgcagtttcaagaaatatataagatcttaaagactcatcaagatgtaggaacagactgtgtaattaatggctcctgagtctacactgccctggtgcattagaaattaaaaatactttgattctaaaaaagcctgaaaatttaatgacttcctgcattaaaaacaactattgggaactgttagtaacaatggctagtcctattagtctctaaatgatccaatgataacctaaatttctcttatttacaaatacagcaattgcaatccagatacacccttaaaatggggcacttaaattgggctccaagactcacagttaaatggaacctaaaattccactctttatccactggtaatagctactttagttccacgccaacagaatacattagactaatggtatgaaaacactagtggacctttattaaagaaaaacatctggacacacacatacatacacctaaaagcttatggacttcattcagggggttagggtaatgtttacaacctattttgtgaaaactgcctgtacaaatcgtcttttaaactaagcattgaaaagttttctcacctagctgggcagtggtggtgcaagcctttaatcccagcacttgggaggcagacgtgggtggaggatctctgagttcaaggccaccgtggttccagacaactagaactacagagaaaccttgtcttgaaaaacccaaaaaattgaaacctaagtagaaatccttgatgtatctagttgtctctgaccaaatgatacattcgttttataaatactaaaattcttttcaggttatggtcaggcatcttatgaaatactattagcaaggaaaacaagaacatgttatctttagtttatgatgcccactgagcaatctcaaaatcataaaagcagtttgatacagtaccggcttgacagtgagtacacactgcctaaatgtgttagcatactttaaaaaaataccctcttatccaactattaggtatcagtgacttaattttttaagaaagatgaccaccactgtagcctttttgaatttgtaaggacaaagaaatgcacctttatccaacatttcaaataaattttaaatggaagatgtttagcactacacactttgtacaaaggaaaacaagcaagaaacagggaaaggggaagcttcctttgtgtgcttttacactgatgtgttctgaaacatggagataatttctggcatatttataagacgactgtatgttctatgtaaataaaaatgctggtgtgcaattgccagtaaaaaaaaaaaaaaaaaaaaaa

SEQ ID NO: 6 - protein, 712 aa, Mus musculus
MEALIPVINKLQDVFNTVGADIIQLPQIVVVGTQSSGKSSVLESLVGRDLLPRGTGVVTRRPLILQLVHVSPEDKRKTTGEENDPATWKNSRHLSKGVEAEEWGKFLHTKNKLYTDFDEIRQEIENETERISGNNKGVSPEPIHLKVFSPNVVNLTLVDLPGMTKVPVGDQPKDIELQIRELILRFISNPNSIILAVTAANTDMATSEALKISREVDPDGRRTLAVITKLDLMDAGTDAMDVLMGRVIPVKLGIIGVVNRSQLDINNKKSVTDSIRDEYAFLQKKYPSLANRNGTKYLARTLNRLLMHHIRDCLPELKTRINVLAAQYQSLLNSYGEPVDDKSATLLQLITKFATEYCNTIEGTAKYIETSELCGGARICYIFHETFGRTLESVDPLGGLNTIDILTAIRNATGPRPALFVPEVSFELLVKRQIKRLEEPSLRCVELVHEEMQRIIQHCSNYSTQELLRFPKLHDAIVEVVTCLLRKRLPVTNEMVHNLVAIELAYINTKHPDFADACGLMNNNIEEQRRNRLARELPSAGSRDKVPSAGGGIGDGGQEPTTGNWRGMLKTSKAEELLAEEKSKPIPIMPASPQKGHAVNLLDVPVPVARKLSAREQRDCEVIERLIKSYFLIVRKNIQDSVPKAVMHFLVNHVKDTLQSELVGQLYKSSLLDDLLTESEDMAQRRKEAADMLKALQGASQIIAEIRETHLW

(19) United States
(12) Patent Application Publication
(10) Pub. No.: US 2014/0155471 A1
(43) Pub. Date: Jun. 5, 2014

(54) TREATING NEUROLOGICAL DISEASE OR INJURY WITH A DYNAMIN-RELATED PROTEIN 1 (DRP1) ENCODING NUCLEIC ACID

(71) Applicants: Kim Tieu, Rochester, NY (US); William J. Bowers, Webster, NY (US); Phillip M. Rappold, Rochester, NY (US)

(72) Inventors: Kim Tieu, Rochester, NY (US); William J. Bowers, Webster, NY (US); Phillip M. Rappold, Rochester, NY (US)

(73) Assignee: UNIVERSITY OF ROCHESTER, Rochester, NY (US)

(21) Appl. No.: 14/131,947

(22) PCT Filed: Jul. 12, 2012
(86) PCT No.: PCT/US12/46465

§ 371 Date: Jan. 10, 2014

Related U.S. Application Data
(60) Provisional application No. 61/506,873, filed on Jul. 12, 2011.

(51) Int. Cl. C12N 15/85

(52) U.S. Cl. CPC C12N 15/85
USPC 514/44 R

(57) ABSTRACT

Provided herein are methods of treating a neurological disease or injury in a subject comprising administering to the subject a recombinant adeno-associated virus (rAAV) vector comprising a DRP1-encoding nucleic acid, wherein the DRP1 encoded by the nucleic acid comprises a mutation compared to wild-type DRP1.

### CROSS REFERENCE TO RELATED APPLICATION

This application claims the benefit of U.S. Application No. 61/506,873 filed Jul. 12, 2011 which is hereby incorporated herein by reference in its entirety.

### FEDERALLY SPONSORED RESEARCH OR DEVELOPMENT

The invention was made with government support under grant numbers ES014899, ES17470 and TL1RR024135 awarded by the National Institutes of Health. The government has certain rights in the invention.

### BACKGROUND

Neurological injuries or disorders have profound clinical effects and, in many cases, result in severe disabilities or reduced life spans in subjects with the injuries or disorders. Parkinson's disease (PD) is the second most common chronic neurodegenerative disorder, after Alzheimer's disease. In the United States alone, about one million people have PD and 50,000-60,000 new cases are diagnosed each year. These figures are expected to increase significantly as the average age of the population increases.

### SUMMARY

Provided herein is a method of treating a neurological disease or injury in a subject comprising administering to the subject a recombinant adeno-associated virus (rAAV) vector comprising a Dynamin-related protein 1 (DRP1) encoding nucleic acid, wherein the DRP1 encoded by the nucleic acid comprises a mutation compared to wild type DRP1.

### DESCRIPTION OF THE DRAWINGS

FIG. 1 shows a diagram of a pFBGR plasmid containing Drp1^{K38A}.

FIG. 2 is a photomicrograph showing that rAAV2 mediates robust expression of Drp1^{K38A}. Drp1^{K38A}-eGFP was packaged in rAAV2 vectors. The right striatum of ten week old C57BL/6 mice was stereotactically infused with 5×10⁹ viral particles. Five weeks later, mice were processed for immunofluorescence against eGFP. As illustrated, Drp1^{K38A} is highly expressed.

FIG. 3A is a graph showing that AAV2-Drp1^{K38A} is protective in the 1-methyl-4-phenyl-1,2,3,5-tetrahydropyridine (MPTP) mouse model of Parkinson's Disease (PD). Ten week old male C57Bl/6 mice were stereotactically injected with AVV2-Drp1^{K38A} into the substantia nigra. Eight weeks later, mice were injected with either MPTP or saline control. Seven days after the last injection, mice were processed for the quantification of dopaminergic (DA) neurons in the substantia nigra. Data represent mean±SEM. N=4-5 mice/group. ^{a}P<0.05 compared to the AAV-GFP saline treated group. ^{b}p<0.05 compared to the AAV-Drp1^{K38A}, MPTP treated group. Data were analyzed by two-way ANOVA followed by Newman-Keuls post-hoc test.

FIG. 3B is a graph showing that AAV2-Drp1^{K38A} is protective in the MPTP mouse model of PD. Ten week-old male C57Bl/6 mice were stereotactically injected with AVV2-Drp1^{K38A} into the substantia nigra. Four weeks later, mice were injected with either MPTP or saline control. Seven days after the last injection, mice were processed for dopamine (DA) terminals in the striatum. Data represent mean±SEM, N=4-5 mice/group. ^{a}P<0.05 compared to the AAV-GFP saline treated group. ^{b}P<0.05 compared to the AAV-Drp1^{K38A}, MPTP treated group. Data were analyzed by two-way ANOVA followed by Newman-Keuls post-hoc test.

FIG. 3C is a graph showing that AAV2-Drp1^{K38A} is protective in the MPTP mouse model of PD. Ten week-old male C57Bl/6 mice were stereotactically injected with AVV2-Drp1^{K38A} into the substantia nigra. Four weeks later, mice were injected with either MPTP or saline control. Seven days after the last injection, mice were processed for levels of striatal DA. Data represent mean±SEM, N=4-5 mice/group. ^{a}P<0.05 compared to the AAV-GFP saline treated group. ^{b}P<0.05 compared to the AAV-Drp1^{K38A}, MPTP treated group. Data were analyzed by two-way ANOVA followed by Newman-Keuls post-hoc test.

FIG. 4 is a photomicrograph showing that in post-mortem human samples (A-E), Drp1 immunoreactivity (dark gray) was significantly higher in nigral dopaminergic neurons (black) of PD patients (for example, in panels B and C, where C is an enlarged neuron from B) than in normal control subjects (panel A). In the cerebellum (panels D and E), the expression of Drp1 in Purkinje and granule neurons was comparable between a PD subject (panel D) and a normal subject (panel E). Scale bars: i, j, l, m=10 μm, k=2 μm. Immunostaining was visualized using 3,3′-diaminobenzidine.

FIG. 5 shows that, using in vivo microdialysis followed by HPLC analysis, mdivi-1 was detected in the striatal dialysate with a peak at 3 hours after an intraperitoneal (i.p.) injection.

FIG. 6 shows the ability of mdivi-1 to restore presynaptic dysfunction in Pink1−/− mice. Twelve month old Pink1−/− and age-matched Pink1+/+ mice were injected i.p. twice daily with either mdivi-1 or vehicle for 3 days followed by in vivo microdialysis to assess depolarization-induced DA overflow in the striatum via perfusion of high-KCl artificial cerebrospinal fluid (aCSF). Pink1−/− mice exhibited significantly less DA overflow compared to their control Pink1+/+ counterparts (a). Simultaneous quantification of serotonin in these dialysate indicates this deficit was specific to DA (b). When treated with mdivi-1, a complete restoration of evoked DA overflow was achieved in these mutant animals (FIG. 6A). Mdivi-1 did not affect the transport activity of DAT (c).

FIG. 7A-D shows that mdivi-1 improved evoked DA overflow in the absence of promoting regeneration of nigral DA neurons terminals or total DA content,

FIG. 8A-C shows that mdivi-1 significantly prevented MPTP induced-loss of dopaminergic cell body terminals and DA content

FIG. 9 shows that Drp1-K38A restores synaptic release of striatal DA. Immunofluorescence revealed robust expression of eGFP (a), Drp1-K38A (b) and hFis1 (c) in nigral DA neurons after 8 weeks of stereotactic delivery of 5×10⁹ rAAV2 particles right above the substantia nigra. For ultrastructural analysis of mitochondria in striatal DA axonal terminals, coronal striatal sections of Pink1+/+(d-f) and Pink1−/− littermates (FIG. 11) transduced with AAV2-GFP (d). AAV2-K38A (e) or AAV2-hFis1 (f) were incubated with anti-tyrosine hydroxylase (TH) antibody, whose immunoreactivity was visualized using 3,3-Diaminobenzidine and subsequently processed for electron microscopy. Arrows indicate axonal terminals positive for TH-containing mitochondria, whereas arrowheads indicate those that reside in other cell types. Measures of mitochondrial size and shape were quantified blindly and grouped into different size bins (g) or expressed as aspect ratio (b, a measurement of major/minor axes as an index of roundness). Fifty clearly identifiable mitochondria were randomly selected per mouse. Data represent mean of three animals. Scale bars: a-c=400 μm, d-f=200 nm. To assess the impact of Pink1 on DA release in vivo, ˜1 yr old Pink1+/+ (WT) and Pink1−/− littermates (KO) were transduced with GFP, Drp1K38A or hFis, as described in the Examples, 8 weeks before in vivo microdialysis was performed in freely moving mice (i.j). To evoke depolarization-induced release of DA, a total of 240 nmoles KCl in isotonic artificial cerebral spinal fluid (aCSF) was delivered through the probe over a 15-min period (shaded box). Striatal dialysates were collected every 15 min and analyzed simultaneously for DA and serotonin levels using HPLC. Areas under the curve were generated using GraphPad Prism® and analyzed by two-way ANOVA followed by Newman-Keuls post-hoc test. n=4-5 mice/group. *P<0.05 compared to the WT group with GFP, #P<0.05 compared to the KO group with GFP. (k) After microdialysis, brains were removed and processed for stereological cell counts of DA neurons, striatal terminal density, and total striatal DA content.

FIG. 10 shows rAAV-mediated gene transfer in nigral DA neurons. rAAV2 encoding eGFP (a), Drp1-K38A (b) or hFis1 (c) were stereotactically infused right above the substantia nigra using a convection enhanced delivery method. Eight weeks after gene delivery, immunofluorescence demonstrated robust expression and co-localization of these proteins in nigral DA neurons. Drp1-K38A expression was evident by the expression of the tagged eGFP, and the appearance of intracellular Drp1 aggregates (characteristic of Drp1-K38A effects) in some DA neurons is illustrated in merged orthogonal images (b). The punctate appearance of hFis1, which was assessed by the expression of the tagged myc, is consistent with the localization of this protein in mitochondria. Scale bar: a-c=20 μm.

FIG. 11 shows ultrastructural analysis of mitochondria in striatal DA axonal terminals of Pink1−/− mice. (a) The size and shape of mitochondria in non-TH positive structures are highly variable ranging from small to highly elongated morphology. Thus, immuno-electron microscopy was developed and utilized to analyze those in DA terminals. Tyrosine hydroxylase immunoreactivity was visualized using 3,3′-Diaminobenzidine. Ultrathin 70 nm-thick sections were cut and counterstained with lead citrate and uranyl acetate. Images were obtained with a Hitachi 7650 TEM with an attached Gatan Erlanshen 11 Megapixel digital camera system. Arrows indicate axonal terminals are positive for TH containing mitochondria and arrowheads indicate those that reside in other cell types. Mitochondria in Pink1−/− mice transduced with Drp1-K38A (c) or hFis1 (d) appeared more elongated or smaller, respectively, as compared to those that received GFP control (b). Scale bars: a=1 μm, b-d=200 nm.

FIG. 12 shows that loss of Pink1 function does not affect synaptic release of serotonin. Striatal dialysates from Pink1−/− and Pink1+/+ littermates (˜12 months old) were collected every 15 min and analyzed for serotonin levels using HPLC. Evoked depolarization-induced release of DA was performed as described in FIG. 1. n=4-5/group.

FIG. 13 shows that Drp1 inhibition protects against active neurodegeneration and pre-synaptic dysfunction in MPTP-treated mice. ˜10 week-old C57Bl/6 mice were stereotactically infused right above the nigra with rAAV2 particles as described in FIG. 1. Eight weeks after gene delivery, mice were injected with MPTP (20 mg/kg, i.p. once daily for 5 days) or saline, and, 7 days after the last injection, mice were processed for stereological cell counting (a), striatal DA terminals (b) and total striatal DA levels (c). n=3-6 per group, analyzed by two-way ANOVA followed by Newman-Keuls post-hoc test. *P<0.05 compared to group receiving MPTP and GFP control; #P<0.05 compared to group receiving Drp1-K38A and saline. (d) ˜10 week-old C57Bl/6 mice were injected with MPTP as described above and seven days after the last MPTP injection, rAAV2 was infused to the nigra for 8 weeks prior to in vivo microdialysis. KCl-evoked DA released was performed as described in FIG. 1. After microdialysis, brains were removed and processed for nigrostriatal pathology (e). n=5 mice/group *P<0.05 compared to the respective saline groups, analyzed by one-way ANOVA.

FIG. 14 shows quantitative measurement of mitochondrial size in a mouse model of Huntington's disease. Electron micrographs of striatal neurons from non-transgenic (A) and transgenic (Tg) R6/2 mice (B). The whole neuron was captured at a magnification of 2,000×. Nuclear aggregates in Tg animals were identified by an antibody against huntingtin protein (arrow). Individual mitochondria were measured using Image-Pro. All mitochondria (20-30) in a given cell and ˜20 cells/animal from more than one striatal section were analyzed. (C) Data represent % of total mitochondria (˜600 from 2 mice/genotype)±SEM, grouped into different size bins and analyzed using t-test. *p<0.05.

FIG. 15 shows that rAAV2-Drp1-K38A attenuates motor deficits in transgenic R6/2 mice. Three week-old transgenic (Tg)-R6/2 mice and non-transgenic (Ntg) littermates were stereotactically injected into both striata using convection enhanced delivery. One week after the surgery, locomotor activities were assessed biweekly using infrared photobeams chambers. Tg-R6/2 mice receiving rAAV2-GFP control (n=2) displayed significant impairment in locomotor movements as compared to the Tg group that received rAAV2-Drp1-K38A (n=3). rAAV2-Drp1-K38A did not affect locomotor function of Ntg mice (n=4) as compared to the Ntg group that received rAAV2-GFP control (n=5). Units expressed as % control of Ntg-rAAV2-GFP group at four weeks old.

FIG. 16 shows that rAAV2-DRP1-K38A attenuates the formation of nuclear aggregates. Viral particles (5×10⁹) of rAAV2-eGFP or rAAV2-DRP1-K38A-eGFP were stereotactically infused into the striatum using convection enhanced delivery in transgenic (Tg) R6/2 mice and their non-transgenic (Ntg) littermates. 10 weeks after gene delivery, immunofluorescence revealed robust expression of eGFP and Drp1-K38A in striatal neurons. More importantly, Drp1-K38A dramatically reduced the formation of nuclear htt aggregates in the transgenic animals.

### DETAILED DESCRIPTION

Provided herein is a method of treating a neurological disease or injury in a subject. The method comprises administering to the subject a recombinant adeno-associated virus (rAAV) vector comprising a DRP1-encoding nucleic acid, wherein the DRP1 encoded by the nucleic acid comprises a mutation compared to wild type DRP1.

Throughout this application, by treating is meant a method of reducing or delaying one or more effects or symptoms of a neurological disease or injury. The subject can be diagnosed with the neurological disease or injury or can be determined to be at risk prior to treatment. Treatment can also refer to a method of reducing the underlying pathology rather than just the symptoms. The effect of the administration to the subject can have the effect of but is not limited to reducing one or more symptoms of the neurological disease or injury, a reduction in the severity of the neurological disease or injury, the complete ablation of the neurological disease or injury, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

In the methods set forth herein, the subject can have or be at risk for a neurological disease or injury. This can be a central nervous system (CNS) injury or disease. CNS injuries include, but are not limited to spinal cord injuries or head injuries. The injury or disease can also be a peripberal nervous system (PNS) injury or disease. These include, but are not limited to peripheral neuropathy and nerve injuries. A neurological injury can also be, and not to be limiting, a surgical injury, a chemical injury, a physical injury, an injury caused by radiation, diabetic neuropathy, an injury related to infection, an injury related to an autoimmune disorder, an injury related to cancer, an injury related to organ failure (for example, heart, renal or liver failure), an injury related to drug toxicity or an injury related to a genetic disease. Thus, the subject at risk for a neurological disease may have a genetic propensity to the disorder, including for example dementia, Huntington's disease, or the like. A subject at risk for neurological injury may have an occupation (e.g. certain military assignments) that puts the subject at risk.

The neurological disease or injury can be a neurological disease or injury that comprises mitochondrial fragmentation or mitochondrial dysfunction. Mitochondria are double-membrane organelles that provide energy to cells and hence play a critical role in cell survival and function. The morphology and function of mitochondria can be maintained and controlled by fission and fusion, which are governed by their respective mitochondrial fission and fusion proteins. Mitochondrial fission leads to multiple smaller mitochondria which are more motile within the cell, therefore, facilitating their sub-cellular distribution. In contrast, the process of fusion results in larger mitochondria, which could offer a larger ATP supply and greater ability to tolerate mitochondrial injury and mutation. The dynamic relationship between fission and fusion also plays a role in regulating mitochondrial-dependent cell death. Consequently, a balance of fusion and fission is important, not only to mitochondrial morphology, but also for function and survival of cell. The neurological disease or injury can also comprise or be associated with a mutation in mitochondrial DNA.

The disease or injury can alter mitochondrial morphology, bioenergetics and/or mitochondrial migration. For example, the neurological disease or injury can be, but is not limited to, Parkinson's disease, Alzheimer's disease, Huntington's disease, amyotrophic lateral sclerosis, stroke, ischemia and neuropathic pain.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

As used throughout, the recombinant adeno-associated (rAAV) vector can comprise a nucleic acid from the genome of any adeno-associated virus serotype. For example, the vector can comprise a nucleic acid from an AAV1, AAV2, AAV3, AAV3B, AAV4, AAV5, AAV6, AAV7, AAV8, AAV9, AAV10 or AAV11 genome. The recombinant vector can also be a pseudotyped vector, comprising nucleic acid sequences from more than one AAV serotype. For example, the rAAV vector can comprise a nucleic acid encoding AAV2 and a nucleic acid encoding one or more capsids from another serotype, for example, from AAV1, AAV5 or AAV8 capsid. See, for example, “Reimsnider et al. “Time course of transgene expression after intrastriatal pseudotyped rAAV2/1, rAAV2/2. rAAV2/5, and rAAV2/8 transduction in the rat,” Mol. Ther. 15(8): 1504-11 (2007). The rAAV vector can also comprise a nucleic acid encoding a capsid sequence(s) from any AAV that has been modified to facilitate vector targeting. For example, a sequence encoding a peptide that targets a particular cell type can be inserted in a nucleic acid encoding a capsid sequence to allow targeting of the vector to a specific cell type or to a cell type that has a different tropism from the tropism of the AAV backbone of the vector. See, for example, Shi et al., “Insertional mutagenesis of the adeno-associated virus type 2 (AAV2) capsid gene and generation of AAV2 vectors targeted to alternative cell-surface receptors,” Hum. Gene Ther. 12: 1697-1711; and Wu et al. “Mutational analysis of the adeno-associated virus type 2 (AAV2) capsid gene and construction of AAV2 vectors with altered tropism,” J. Virol. 74: 8635-8647 (2000). An AAV capsid sequence can also be modified to encode an antibody or a fragment thereof that recognizes a cell surface marker. An AAV capsid sequence can also be modified to encode a ligand that recognizes a cell surface receptor in order to direct delivery of the vector to specific cell types. See, for example, Yang et al. “Development of novel cell surface CD34-targeted recombinant adenoassociated virus vectors for gene therapy,” Hum. Gene Ther. 9(13): 1929-37 (1998).

As set forth above, the rAAV vector comprises a dynamin-related protein (DRP1) encoding nucleic acid, wherein the DRP1 encoded by the nucleic acid comprises a mutation compared to wild type DRP1. DRP1 is also known as DLP1; DVLP; VPS1; DYMPLE: HDYNIV; DYNIV-11; FLJ41912. An example of a nucleic acid sequence that encodes wild type human DRP1 is provided under GenBank Accession No. NM_{—}012062.3 and is set forth herein as SEQ ID NO: 1. SEQ ID NO: 1 encodes the DRP1 protein sequence provided under GenBank Accession No. NP_{—}036192.2 that is set forth herein as SEQ ID NO: 2. Another example is a nucleic acid sequence that encodes wild type rat DRP1, which is provided under GenBank Accession No. NM_{—}053655 and is set forth herein as SEQ ID NO: 3. SEQ ID NO: 3 encodes the rat DRP1 protein sequence provided under GenBank Accession No. NP_{—}446107 that is set forth herein as SEQ ID NO: 4. Another example is a nucleic acid sequence that encodes wild type mouse DRP1 is provided under GenBank Accession No. NM_{—}152816.2 and is set forth herein as SEQ ID NO: 5. SEQ ID NO: 5 encodes the mouse DRP1 protein sequence provided under GenBank Accession No. NP_{—}690029.2 that is set forth herein as SEQ ID NO: 6.

The mutation in DRP1 can be one or more mutations selected from the group consisting of K38A (replacement of lysine at position 38 of SEQ ID NO: 2 or SEQ ID NO: 4 with alanine), G350D (replacement of glycine at position 350 of SEQ ID NO: 2 or SEQ ID NO: 4 with aspartic acid), G363D (replacement of glycine at position 363 of SEQ ID NO: 2 or SEQ ID NO: 4 with aspartic acid), A395D (replacement of alanine at position 395 of SEQ ID NO: 2 or SEQ ID NO: 4 with aspartic acid), D225N (replacement of aspartic acid at position 225 of SEQ ID NO: 2 or SEQ ID NO: 4 with asparagine) and D231 N (replacement of aspartic acid at position 231 of SEQ ID NO: 6 with asparagine). See Chang-Rung et al. “A Lethal de Novo Mutation in the Middle Domain of the Dynamin-related GTPase Drp1 Impairs Higher Order Assembly and Mitochondrial Division,” J. Biol. Chem. 285(42): 32494-32503 (2010): Pitts et al. “The Dynamin-like Protein DLP1 is Essential for Normal Distribution and Morphology of the Endoplasmic Reticulum and Mitochondria in Mammalian Cells,” Molecular Biology of the Cell 10: 440304417 (1999); and Smimova et al. “Dynamin-related Protein Drp1 is Required for Mitochondrial Division in Mammalian Cells,” Molecular Biology of the Cell 12: 2245-2256 (2001). These mutations are not meant to be limiting, as one of skill in the art could make any desired mutation, for example, a substitution (including, for example, a conservative substitution), an insertion or a deletion in DRP1, and utilize cell based assays or animal models to assess the ability of the mutant DRP1 to inhibit mitochondrial fragmentation. As set forth in the Examples, the MPTP mouse model of Parkinson's Disease can also be utilized to assess the ability of a mutant DRP1 to protect dopaminergic neurons.

The rAAV vector can comprise a plasmid wherein the plasmid comprises a promoter functionally linked to the DRP1 encoding nucleic acid. The plasmid can be any plasmid that is compatible with an AAV vector, for example, a pFBGR plasmid, as described in the Examples.

The promoter can be any desired promoter, selected by known considerations, such as the level of expression of a nucleic acid functionally linked to the promoter and the cell type in which the vector is to be used. That is, the promoter can be tissue/cell-specific to promote expression of the nucleic acid in specific cells, tissues or organs. Promoters can be prokaryotic, eukaryotic, fungal, nuclear, mitochondrial, viral or plant promoters. Promoters can be exogenous or endogenous to the cell type being transduced by the vector. Promoters can include, for example, bacterial promoters, known strong promoters such as SV40 or an AAV promoter from any AAV serotype, such as an AAV p5 promoter, an AAV p19 promoter or an AAVp40 promoter. Other promoters include promoters derived from actin genes, immunoglobulin genes, cytomegalovirus (CMV), adenovirus or bovine papilloma virus. Adenoviral promoters, such as the adenoviral major late promoter can also be utilized. Other promoters include inducible heat shock promoters, promoters derived from respiratory syncytial virus and promoters derived from Rous sarcomas virus (RSV). An inducible promoter such as the tetracycline inducible promoter or a glucocorticoid inducible promoter can also be utilized. Any regulatable promoter, such as a metallothionein promoter or a heat-shock promoter can also be used. Furthermore, a Cre-loxP inducible system can be utilized, as well as the Flp recombinase inducible promoter system. Additional examples of promoters include, but are not limited to, a glial fibrillary acidic protein (GFAP) promoter, a neuronal specific nuclear protein (NeuN) promoter, a F4/80 promoter, a ROSA promoter or a prion protein promoter.

The rAAV vector can comprise at least two AAV inverted terminal repeats (ITRs). The ITRs can flank the nucleic acid encoding DRP1. The ITRs can also flank a plasmid comprising a DRP1 encoding nucleic acid. By “adeno-associated virus inverted terminal repeats” or “AAV ITRs” is meant the art-recognized regions found at each end of the AAV genome, which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The AAV ITR can be derived from any of several AAV serotypes, including without limitation, AAV1, AAV2, AAV3, AAV3B, AAV4, AAV5, AAV6, AAV7, AAV8, AAV9, AAV10 or AAV11. Furthermore, 5′ and 3′ ITRs, which flank a selected heterologous nucleotide sequence in an AAV vector, for example, a nucleotide sequence encoding a mutant DRP1, need not be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Thus, the ITRs can be from the same serotype as the backbone of the rAAV vector or from a different serotype. For examples, the rAAV vector can be a recombinant AAV2 vector comprising AAV2ITRs, an AAV2 vector comprising an AAV2 ITR and an AAV5 ITR, an AAV2 vector comprising AAV5 ITRs, a recombinant AAV2 vector comprising AAV8 ITRs, a recombinant AAV5 vector comprising AAV2 ITRs, etc. A vector with an AAV backbone and ITRs can be constructed that is appropriate for adequate gene expression in the desired cell, tissue or organism being transduced with the vector.

The rAAV vector set forth herein can be in a viral particle or virion. An rAAV virion is an infectious, replication-defective virus composed of an AAV protein shell, encapsidating a heterologous nucleotide sequence of interest, for example, a mutant DRP1 protein, which is flanked on both sides by ITRs. A rAAV virion is produced in a suitable host cell with an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

Methods of delivery of viral vectors include, but are not limited to, intra-arterial, intra-muscular, intravenous, intranasal and oral routes. Generally, rAAV virions may be introduced into cells of the CNS using either in vivo or ex vivo transduction techniques. For in vivo delivery, the rAAV virions can be administered via injection, intraventricular administration, lumbar puncture, grafting, cannulation, stereotactic administration or convection enhanced delivery (CED), to name a few. In vivo delivery also encompasses delivery at a surgical site. The rAAV virion can be delivered to a brain region, for example, to the substantia nigra, the striatum or the hippocampus, for example, when surgery is otherwise required.

Any convection-enhanced delivery device (CED) method is appropriate for delivery of viral vectors. The form of delivery can be performed using an infusion pump, which is commercially available from a variety of suppliers, for example, from World Precision Instruments, Inc. (Sarasota, Fla.). A viral vector can be delivered via a catheter, cannula or other injection device that is inserted into CNS tissue (intraparenchymally, intraventricularly, intravascularly, subdurally, epidurally or intrathecally) in the chosen subject. One of skill in the art can readily determine which general area of the CNS is an appropriate target. For example, and not to be limiting, when treating PD, the striatum or substantia nigra are suitable areas of the brain to target. Stereotactic maps and positioning devices are available, for example from ASI Instruments (Warren, Mich.). Positioning may also be conducted by using anatomical maps obtained by CT and/or MRI imaging of the subject's brain to help guide the injection device to the chosen target. Once the device is adequately positioned, an effective amount of the rAAV can be delivered.

According to the methods taught herein, the subject is administered an effective amount of the rAAV. The terms effective amount and effective dosage are used interchangeably. The term effective amount is defined as any amount necessary to produce a desired physiologic response. For example, a composition comprising about 10², 10³, 10⁴, 10⁵, 10⁶, 10⁷, 10⁸, 10⁹, 10¹⁰, 10¹¹, 10¹² rAAV virions or any amount of virions in between can be delivered. Effective amounts and schedules for administering the rAAV can be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the type of AAV vector, heterologous nucleic acid, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days.

Pharmaceutical compositions will comprise sufficient rAAV virions to produce a therapeutically effective amount of the mutant DRP1, i.e., an amount sufficient to reduce or ameliorate symptoms of a neurological disease or injury or an amount sufficient to confer the desired benefit. Thus, provided herein is a pharmaceutical composition comprising an effective amount of the rAAV in a pharmaceutically acceptable carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

If transduced ex vivo, the desired recipient cell can be removed from the subject, transduced with rAAV virions and reintroduced into the subject. Alternatively, syngeneic or xenogeneic cells, that do not generate an inappropriate immune response in the subject can be used.

Suitable methods for the delivery and introduction of transduced cells into a subject have been described. For example, cells can be transduced in vitro by contacting AAV virions with CNS cells in appropriate media. Cells comprising the DNA of interest can be identified by utilizing Southern blots and/or PCR, or by using selectable markers. Transduced cells can then be formulated into a pharmaceutical composition and introduced into the subject by various techniques, such as by grafting, injection, cannulation or convection enhanced delivery. Transduced cells can also be administered at a surgical site.

A neural stem cell or a population of neural stem cells (e.g., a stem cell capable of giving rise to neurons, glial cells (e.g. oligodendrocytes) or both) can be transduced with the rAAV virions described herein and administered to a subject with a neurological disorder or injury. Neural stem cells include pluripotent or totipotent stem cells. Such stem cells can be derived from the same subject, or a different subject, including an embryonic subject. Alternatively, the cells can be induced pluripotent stem cells or induced totipotent stem cells.

The number of stem cells to be administered depends on the type of cell; species, age, or weight of the subject; and the extent or type of the injury or disease. Optionally, administered doses range from about 10³-10⁸, including 10³-10⁵, 10⁵-10⁸, 10⁴-10⁷, cells or any amount in between in total for an adult subject. Cells can generally be administered at concentrations of about 5-50,000 cells/microliter. Optionally, administration can occur in volumes up to about 15 microliters per administration site. However, administration to the central nervous system can involve much larger volumes. The method can further comprise administering a therapeutic agent, for example, an agent utilized to treat spinal cord injury or CNS lesions. For example, several agents have been applied to acute spinal cord injury (SCI) management and CNS lesions that can be used in combination with stem cell transplantation. Such agents include agents that reduce edema and/or the inflammatory response. Exemplary agents include, but are not limited to, steroids, such as methylprednisolone; inhibitors of lipid peroxidation, such astirilazad mesylate (lazaroid); and antioxidants, such as cyclosporin A, EPC-Kl, melatonin and high-dose naloxone. These agents can be administered prior to administration of the stem cells, concurrently with the stem cells or subsequent to administration of the stem cells. Thus, the compositions including stem cells can further comprise methylprednisolone, tirilazad mesylate, cyclosporin A, EPC-Kl, melatonin, or high-dose naloxone or any combination thereof. Other therapeutic agents that could be administered prior to, concurrently with or after stem cells include tissue plasminogen activator, prolactin, progesterone, growth factors, etc.

An agent or agents delivered in combination with the cells can be administered in vitro or in vivo in a pharmaceutically acceptable carrier. A pharmaceutically acceptable carrier for the agent can be a solid, semi-solid, or liquid material that can act as a vehicle, carrier or medium. Thus, compositions can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release after administration by employing procedures known in the art. In addition to the representative formulations described below, other suitable formulations for use in a pharmaceutical composition can be found in Remington: The Science and Practice of Pharmacy (21th ed.) ed. David B. Troy. Lippincott Williams & Wilkins, 2005.

Liquid formulations for oral administration or for injection generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. Compositions for inhalation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner. Another formulation that is optionally employed in the methods of the present disclosure includes transdermal delivery devices (e.g., patches). Such transdermal patches may be used to provide continuous or discontinuous infusion of an agent described herein.

The disclosure also provides a pharmaceutical pack or kit comprising one or more containers filled with one or more of the ingredients of the pharmaceutical compositions and/or a delivery means. Instructions for use of the composition can also be included.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. A number of embodiments have been described. Nevertheless, it will be understood that various modifications may be made. Accordingly, other embodiments are within the scope of the following claims.

### EXAMPLES

### Example I

### Human Samples

Human paraffin-embedded sections (7 μm) were obtained from the Parkinson Brain Bank at Columbia University and immunohistochemistry was performed using a polyclonal antibody against Drp1 (1:100. BD Biosciences, Franklin Lake, N.J.). Immunostaining was visualized using 3,3′-diaminobenzidine with cobalt/nickel enhancement.

### Mdivi-1 Preparation.

Mdivi-1 (3-(2,4-dichloro-5-methoxyphenyl)-2-sulfanyl-4(3H)-quinazolinone) was purchased from Enzo Life Sciences International, Inc. (Farmingdale, N.Y.) and dissolved in DMSO (100 mg/mL) as a stock solution. For injections, mdivi-1 was diluted in sterile saline (1% DMSO). Each mdivi-1 dose was gently sonicated (Model S3000 Sonicator with tapered microtip; Misonix, Inc., Farmingdale, N.Y.) at a power level of 0.5-1 for 30s producing a homogenous suspension and injected intraperitoneally (i.p.) immediately. For cell culture experiments, mdivi-1 stock solution was diluted in culture medium to varying working concentrations.

### MPTP and Mdivi-1 Treatments.

For all studies, 10-12 week old male C57BL/6 mice were randomly assigned to receive intraperitoneal injections of either MPTP (25 mg/kg, Sigma, St. Louis, Mo.) or saline once daily for 5 days. For the neuroprotection studies, mice received twice daily i.p. injections (20 mg/kg) with mdivi-1 beginning on the day of the first MPTP injection and continued until mice were sacrificed 7 days after last MPTP injection. For the neurorescue studies, mice received twice daily i.p. injections (20 mg/kg) with mdivi-1 beginning 7 days after the last MPTP injection and continued for a total of 3 days. To maximize the data yielded from each animal, mice were sacrificed by decapitation and the freshly removed brains were divided into 3 pieces for separate measures of nigrostriatal damage. Upon removal, brains were first divided into rostral and caudal sections via a coronal cut ˜1-2 mm caudal to the optic chiasm. The caudal portion containing the midbrain was immediately placed in 4% paraformaldehyde (4% PFA) for 24 hours. The rostral portion containing the striatum was then divided mid-sagittally into right and left halves. Randomly, one half was placed in 4% PFA for 24 hours, while the other was processed for HPLC analysis of total striatal dopamine. After 24 hours in 4% PFA, tissue was cryoprotected in successive 15% and 30% sucrose phosphate buffer for 2 days then frozen at −80° C. for immunohistochemical studies.

### Stereological Nigral Cell Count and Striatal Optical Density.

Brains from saline and MPTP treated mice were sectioned (30 μm) and processed for stereological cell counts using the optical fractionator method as described 35. Striatal optical densities of TH immunoreactivity were also quantified.

### Measurements of MPTP Metabolism.

To assess whether mdivi-1 treatment interferes with the conversion of MPTP into MPP+, 10-12 week old male C57BL/6 mice received a single i.p. injection of mdivi-1 (20 mg/kg) or vehicle followed immediately by a single i.p. injection of MPTP (25 mg/kg). All mice were killed 90 min after the injections. Striatal tissue levels of MPP+ were measured using HPLC.

### In Vivo Microdialysis.

Stereotactic implantation of guide cannula was performed under ketamine/xylazine (65/6 mg/kg i.p.) anesthesia using the following striatal coordinates, relative to bregma: anterior-posterior +0.5 mm, lateral −2.0 mm, dorsal-ventral −1.5 mm (from surface of brain). Twenty-four hours after recovery from surgery, a microdialysis probe (2-mm membrane, Bioanalytical Systems, Inc., West Lafayette, Ind.) was inserted into the guide cannula and connected to a low torque-dual channel swivel (Instech Laboratories, Inc., Plymouth Meeting, Pa.) which was connected to a syringe pump perfusing with artificial cerebrospinal fluid (aCSF) at 2 μL/min for all studies except mdivi-1 pharmacokinetic studies where the flow rate was 1 μL/min. After a 2-h equilibration period, dialysates were collected every 15 min for all dopamine release studies and every 30 min for mdivi-1 pharmacokinetic studies. Two baseline fractions were collected, after which the perfusate was switched to aCSF containing 100 m KCl (with equimolar reduction in NaCl to maintain osmolality) for 15 min, followed by a return to normal aCSF for an additional hour. Histological examination subsequent to the experiments was performed to verify the placement of the probe in each animal. Samples from the same animals were measured for the contents of mdivi-1, MPP+, DA, and its metabolites. Levels of these molecules and the amount of KCl (delivered to the striatum) were calculated on the basis of the standard curves, probe efficiency (˜8%), flow rate, and duration of sample collection as described in Cui et al. “The organic cation transporter-3 is a pivotal modulator of neurodegeneration in the nigrostriatal dopaminergic pathway,” PNAS USA 106: 8043-8048 (2009).

### Measurements of Striatal DA and MPP+ Levels.

A 12-channel CoulArray® (ESA Inc., Chelmsford, Mass.) equipped with a highly sensitive amperometric microbore cell (model 5041, ESA Inc.) was used to analyze the content of DA and its metabolites with the cell potential set at +220 mV as described in Cui et al. For measurements of total striatal DA content, mice were sacrificed and their striata were dissected out and stored at −80° C. until analysis. On the day of the assay, striatal tissues were sonicated in 50 volumes (wt/vol) of 5% trichloroacetic acid containing 50 ng/ml dihydrobenzylamine as an internal standard. After centrifugation at 15,000 g for 15 minutes at 4° C. the supernatant was removed for HPLC analysis. Briefly, 20 μL samples of dialysates or tissue homogenates were injected manually into a sample injector (with 20 μL sample loop) and eluted on a narrowbore (ID: 2 mm) reverse-phase C18 column (MD-150, ESA, Inc.) using MD-TM (ESA, Inc.) mobile phase (for striatal homogenates pH was adjusted to 4.25). For mdivi-1 pharmacokinetic studies, 20 μL samples were used for mdivi-1 measurement using a UV detector (model no. 526, ESA Inc.) at 298 nm. Samples were injected manually and separated by a narrowbore column (ID: 2.1 mm, Altima HP C18, Alitech Associates, Inc, Deerfield, Ill.) using mobile phases consisting of 35 mM KH2PO4 and 45% acetonitrile, pH 3.2. The flow rate was set at 0.2 mL/min for catecholamines and 0.4 mL/min for mdivi-1 by using a solvent delivery pump (Model 585, ESA Inc.). Peaks were detected by an ESA 8 Channel CoulArray® system. Data were collected and processed using the CoulArray® data analysis program.

Transport Studies.

EM4 cells and human embryonic kidney (HEK 293) cells stably transfected with macrophage scavenger to increase their adherence to tissue culture plastic, overexpressing mouse dopamine transporter or empty vector control were grown in 24-well plates. These cells were washed twice and then preincubated for 20 min at 37° C. in Krebs Ringer Hepes (KRH) buffer (125 mM NaCl, 25 mM HEPES, 5.6 mM glucose, 4.8 mM KCl, 1.2 mM KH2PO4, 1.2 mM CaCl2, 1.2 mM MgSO4, pH 7.4), in the presence or absence of mdivi-1 (1, or 10 μM) or GBR12909 (1 μM). This buffer was then replaced with KRH plus or minus MPP+ (200 μM) or dopamine (100 μM), in the presence or absence of mdivi-1 (1, or 10 μM) or GBR12909 (1 μM) for 30 min. To stop the reaction, cells were rinsed with ice-cold buffer and then immediately removed in 5% trichloroacetic acid, sonicated and centrifuged at 15,000 at 4° C. for min. Supernatant was collected for MPP+ and DA quantification using HPLC. Cell pellet was measured for protein concentration using the BCA assay.

Use of rAAV in Neurological Disease

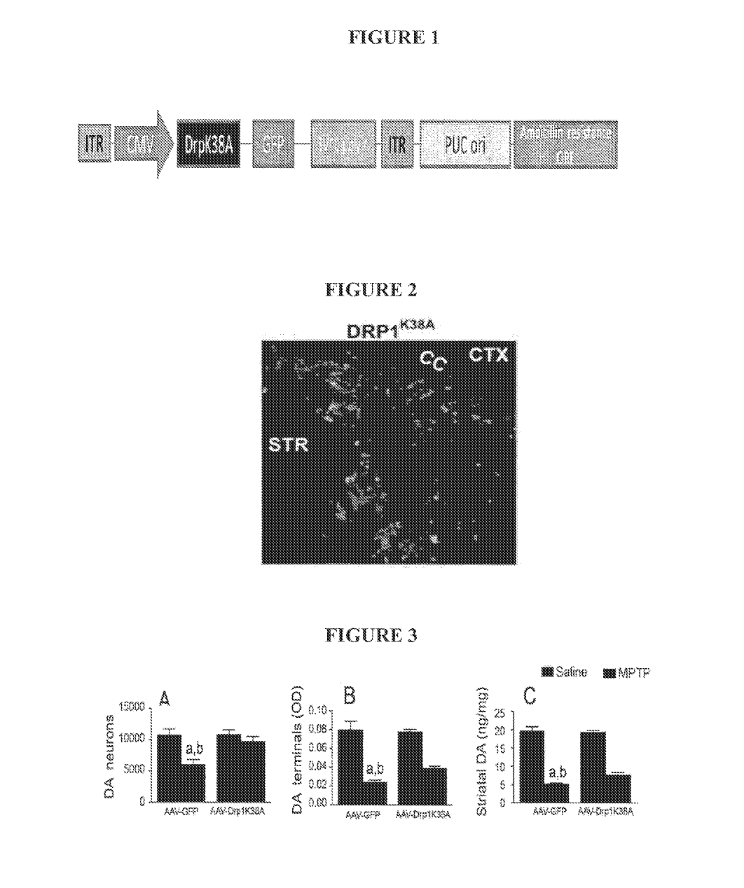

Provided herein are data showing that gene-based applications to block mitochondrial fission are beneficial in animal models of PD. To develop a gene therapy for this approach, recombinant adeno-associated virus (rAAV2) was used to deliver the gene Drp1^{K38A} in order to disable the function of the mitochondrial fission protein Drp1. To generate this viral vector, briefly, Drp1 was tagged with GFP at the C-terminus, using standard molecular biology techniques. These constructs were first cloned into the pBSFBRmcs shuttle vector and then subsequently into a modified pFBGR plasmid backbone. As shown below in FIG. 1, the pFBGR plasmid harbors a cytomegalovirus promoter driven Drp1^{K38A}-GFP gene flanked by inverted terminal repeats. These plasmids were then packaged in rAAV2 vectors. Vector construction and packaging methodology are well established. See, for example, Bowers et al. “Efficacy of adenoviral p53 delivery with SCH58500 in the intracranial 9I and RG2 models,” Ann NY Acad. Sci. 1003: 419-21 (2003); and Bowers et al. “Gene therapeutic strategies for neuroprotection: implications for Parkinson's Disease,” 144(1): 58-68 (1997). When delivered to the mouse brain, Drp1^{K38A} is highly expressed (FIG. 2) demonstrating that this viral vector is effective.

The right striatum and substantia nigra often week old C57BL/6 mice were stereotactically infused with 5×10⁹ viral particles. Four weeks later, mice were processed for immunofluorescence against eGFP. Drp1^{K38A} is highly expressed in nigral and striatal neurons. Most dopaminergic neurons were transduced with Drp1^{K38A} as evidenced by the expression of the tagged eGFP and the appearance of intracellular aggregates. This is characteristic of Drp1^{K38A} effects due to Drp1 aggregation.

To assess the effectiveness of preventing cell death in an animal model of PD, rAAV2 carrying the gene of interest (Drp1^{K38A}) was delivered to the substantia nigra, a brain region that is affected in PD. After four weeks, to allow sufficient time for expression of Drp1^{K38A}, mice were injected with 1-methyl-4-phenyl-1,2,3,6-tetrahydropyridine (MPTP), a neurotoxic molecule that is utilized to model PD by killing dopaminergic neurons that are affected in PD. As seen in FIG. 3, in the group of mice that received the control AAV-GFP, there was a significant loss of dopaminergic neurons (A) and their associated terminals (B). This damage led to the reduction in dopamine (C), a neurotransmitter that is critical for body movement. In the group of animals that received AAV2-Drp1K38A, the neurodegeneration induced by MPTP was significantly reduced. These results show that this approach is a novel treatment for PD.

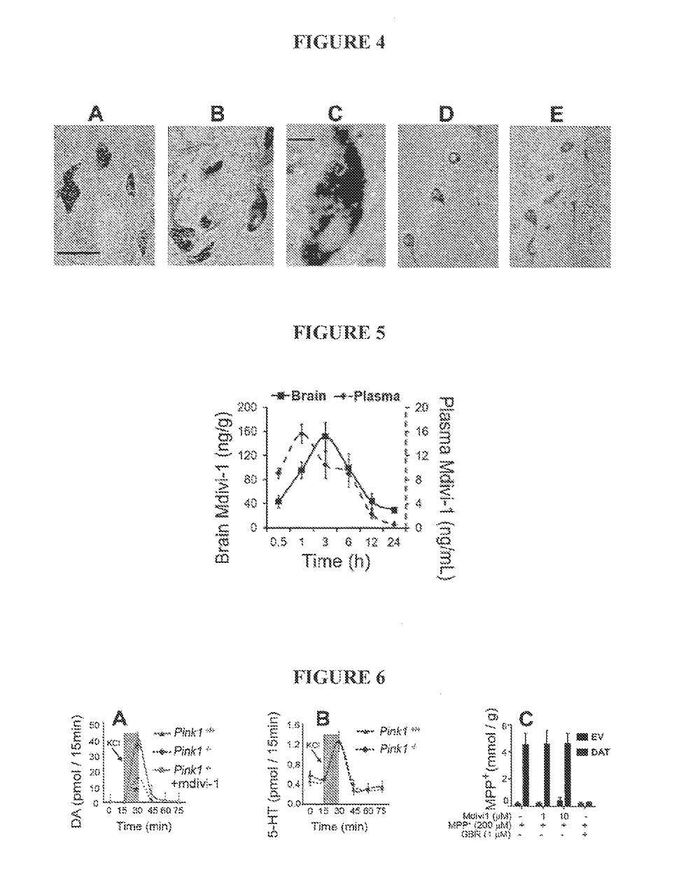

To further demonstrate the importance of targeting Drp1 in humans, the expression of this mitochondrial protein in post-mortem samples of PD and age-matched normal controls (FIG. 4) was examined. Immunohistochemical results indicate the expression of Drp1 was low in nigral dopaminergic neurons in control subjects but dramatically increased in the remaining dopaminergic neurons in PD patients. This difference was not apparent in cerebella neurons, the cell types that are not affected in PD. These human data further support reducing the excessive function of Drp1 in PD.

### Use of Drp1 Inhibitor in Neurological Disease.

The ability of Mitochondrial Division Inhibitor-1 (mdivi-1), a Drp1 inhibitor, to cross the blood-brain barrier was assessed. Using in vivo microdialysis followed by HPLC analysis, mdivi-1 was detected in the striatal dialysate with a peak at 3 hours after an intraperitoneal (i.p.) injection (FIG. 5). Next, the ability of mdivi-1 to restore presynaptic dysfunction in Pink1−/− mice was assessed. These mice have been shown to exhibit impaired mitochondrial function and reduced evoked dopamine (DA) release in acute brain slices. Twelve month old Pink1−/− and age-matched Pink1+/+ mice were injected i.p. twice daily with either mdivi-1 or vehicle for 3 days followed by in vivo microdialysis to assess depolarization-induced DA overflow in the striatum via perfusion of high-KCl artificial cerebrospinal fluid (aCSF). Pink1−/− mice exhibited significantly less DA overflow compared to their control Pink1+/+ counterparts (FIG. 6A). Simultaneous quantification of serotonin in these dialysate indicates this deficit was specific to DA (FIG. 6B). Reduced DA overflow in Pink1−/− mice was not a result of reduced presynaptic dopamine stores as the total striatal DA content and number of nigral DA neurons was normal in these mice. Additionally, because the reduced DA overflow in Pink1−/− was not due to increased dopamine transporter (DAT) activity, the observation that Pink1−/− mice exhibited significantly less DA overflow compared to their control Pink1+/+ counterparts provides the first in vivo evidence of impaired exocytotic release of DA in mice with loss of Pink1 function.

When treated with mdivi-1, however, a complete restoration of evoked DA overflow was achieved in these mutant animals (FIG. 6A). Mdivi-1 treatment did not alter the level of evoked DA release in Pink1+/+ mice showing that this molecule alone does not promote DA release. To test whether this enhanced DA overflow was due to mdivi-1 induced DA reuptake inhibition, stable cells overexpressing DAT in the presence of its substrates 1-methyl-4-phenylpyridinium (MPP+) or DA were utilized. Mdivi-1 did not affect the transport activity of DAT (FIG. 6C). Together, these results indicate that mdivi-1 is capable of correcting pre-existing dopaminergic synaptic dysfunction in Pink1−/− mice, through its established mitochondrial fusion-promoting effect. Due to the lack of overt neurodegeneration in Pink1−/− mice, mice were injected with MPTP using a subacute regimen that produces ˜70% loss of striatal DA and ˜40-550% loss of DA neurons. To more closely model human scenario, the lesion was allowed to stabilize for seven days after the last MPTP injection before mdivi-1 was administered i.p. twice daily for 3 days. Mdivi-1 improved evoked DA overflow in the absence of promoting regeneration of nigral DA neurons terminals or total DA content (FIG. 7A-D).

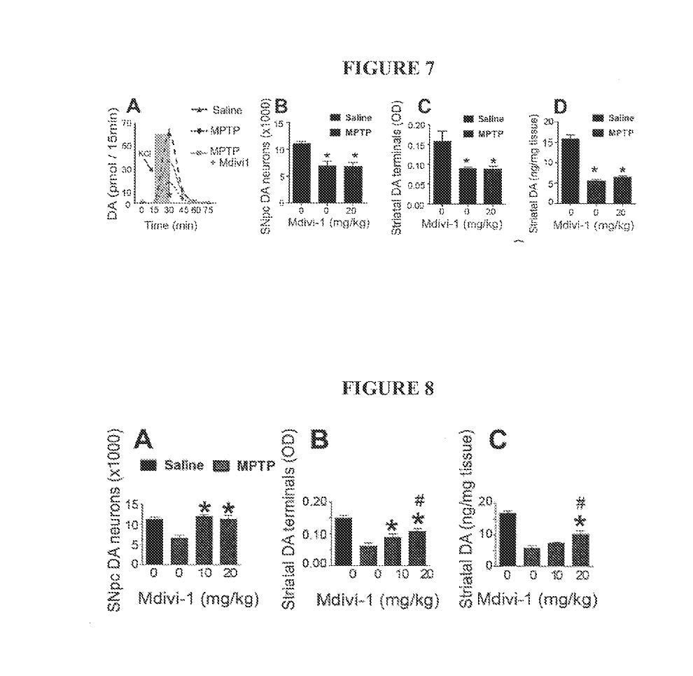

To determine the efficacy of mdivi-1 in the setting of active neurodegeneration, this small molecule was delivered together with MPTP. Mdivi-1 significantly prevented MPTP induced-loss of dopaminergic cell body terminals and DA content (FIG. 8A-C). Mdivi-1 did not interfere with the levels of MPP+ in the brain as evidenced by the observations that striatal levels of MPP+ 90 minutes after MPTP injection in C57Bl mice did not differ between the group that receive mdivi-1 (10.0±0.34 μg MPP+/g striatal tissue, n=4) and vehicle control (9.95±1.07 μg MPP+/g striatal tissue, n=5).

To further determine the relevance of targeting Drp1 in humans, the expression of this mitochondrial protein was assessed in post-mortem samples of PD and age-matched normal controls. Immunohistochemical results indicate the expression of Drp1 was low in DA neurons in control subject but dramatically increased in the remaining DA neurons in PD patients. This difference was not apparent in cerebella neurons, the cell types that are not affected in PD.

### Example 2

### Plasmids

Drp1^{K38A}, eGFP, and hFis plasmids have been described in Cui et al. (J. Biol. Chem. 285:11740-11752 (2010). To monitor the expression of these proteins after rAAV injections, Drp1^{K38A} was tagged with eGFP and hFis with myc at the C-terminus using standard molecular biology techniques. Drp1^{K38A}-eGFP and hFis-myc, eGFP constructs were first cloned into the pBSFBRmcs shuttle vector and then subsequently into a modified pFBGR plasmid backbone devoid of the eGFP gene. The pFBGR plasmid harbors a cytomegalovirus promoter driven enhanced green fluorescent protein (eGFP) gene flanked by inverted terminal repeats. These plasmids were transiently transfected into baby hamster kidney cells and transgene expression was confirmed by immunocytochemistry before viral packaging. These procedures are described in Janelsins et al. (Am. J. Pathol. 173:1768-1782).

rAAV Packaging:

Briefly, rAAV2 was produced by co-infecting cultures of SF9 cells at log phase (2×10⁶ cells/ml) with passage 2 baculovirus of pFBDAAV (serotype viral proteins), and pFBDLSR (Rep 52 & Rep 72) and pFB-Drp1^{K38A}-eGFP, pFB-hFis1-myc or pFB-eGFP at a MOI=5 each. Cultures were incubated 72 h at 28° C. and harvested by centrifugation. Pelleted cells were resuspended in PBS with MgCl₂, serially frozen at −70° C. and thawed at 37° C. three times. The lysates were centrifuged and optical grade CsCl₂ (Shelton Scientific) was added to supernatant; final concentration was confirmed by refractory index. rAAV particles were handed on a CsCl₂ gradient by ultracentrifugation. Fractions with a refractory index of 1.372, corresponding to the position of viable viral particles, were collected and subsequently dialyzed against PBS. AAV particles were titered, relative to rAAV-eGFP titers that were packaged in parallel, by transduction assay and PCR-based enumeration of genome-containing AAV particles.

Stereotactic injections of rAAV2 via convention enhanced delivery. C57BL/6 male mice (10-12 weeks old) or Pink1-null mice and wild type littermates (˜1 year old) received bilateral stereotactic injections of rAAV2 capsids right above the substantia nigra in accordance with approved University of Rochester animal use guidelines. Under Avertin® anesthesia (300 mg/kg), mice were positioned in a stereotactic apparatus and an incision was made to expose bregma on the skull. Two burr holes were drilled bilaterally over the injection coordinates (relative to bregma: −3.1 mm caudal, +1.3 mm lateral, −4.2 mm ventral). The injection set up consisted of a frame-mounted micromanipulator, holding an UltraMicro pump (WPI Instruments, Sarasota, Fla.) with a Hamilton syringe and a 33 GA needle (Hamilton, Reno, Nev.). The needle was lowered into the parenchyma at a rate of 0.8 mm/minute, and then held in place for 2 minutes before injection. rAAV2 vectors (5×10⁹) transducing units were delivered to each side of the substantia nigra in a 5 μl volume. rAAV2 capsids were delivered by convection enhanced delivery (a method to augment the distribution of molecules delivered into the brain by sustaining a pressure gradient for the duration of the injection) by using increasing step-wise injection rates of 100 nl/minute for 6 minutes, 200 nl/minute for 10 minutes, and 400 nl/minute for 6 minutes. After injection, the needle was allowed to rest in place for 2 minutes, then withdrawn at a rate of 0.4 mm/minute. Incisions were sutured with 4-0 Vicryl (Ethicon, Inc., Cornelia, Ga.), triple antibiotic and lidocaine topical ointments were applied, and mice placed in a recovery chamber at 37° C. overnight. Four weeks later, mice were randomly assigned to receive either MPTP or saline.

### MPTP Treatment.

For all studies, 10-12 week old male C57BL/6 mice were randomly assigned to receive i.p. injections of either MPTP (20 mg/kg, Sigma) or saline once daily for 5 days. For neuroprotection studies, MPTP injections began 4 weeks after AAV delivery and mice were sacrificed 7 days after the last MPTP dose. For neuro-rescue studies. AAV was delivered 7 days after the last MPTP injection and mice were sacrificed 6 weeks after AAV delivery. To maximize the data yielded from each animal, mice were sacrificed by decapitation and the freshly removed brains were divided into 3 pieces for separate measures of nigrostriatal damage. Upon removal, brains were first divided into rostral and caudal sections via a coronal cut ˜1-2 mm caudal to the optic chiasm. The caudal portion containing the midbrain was immediately placed in 4% paraformaldehyde (PFA) for 24 hours. The rostral portion containing the striatum was then divided mid-sagittally into right and left halves. Randomly, one half was placed in 4% PFA for 24 hours, while the other was processed for HPLC analysis of total striatal dopamine. After 24 hours in 4% PFA, tissue was cryoprotected in successive 15% and 30% sucrose phosphate buffer for 2 days then frozen at −80° C. for immunohistochemical studies.

### Immunostaining and Colocalization.

Coronal brain sections (30 μm) from mice receiving rAAV2 were incubated in M.O.M™ mouse IgG blocking reagent (Vector Laboratories, Burlingame, Calif.) overnight before incubation with polyclonal anti-eGFP (1:500, Invitrogen) and monoclonal antibodies against tyrosine hydroxylase (1:500; Calbiochem. Darmstadt, Germany), For hFis1, monoclonal antibody against myc (9E10, Sigma, St. Louis, Mo.) and TH polyclonal clonal antibody (Calbiochem. Darmstadt, Germany) were used. Corresponding secondary antibodies Alexa Fluor 488 and 594 (Invitrogen, Carlsbad, Calif.) were used. Images were scanned at 0.5 μm intervals throughout the whole section and analyzed using confocal microscopy (FV 1000; Olympus, Center Valley, Pa.).

### Stereological SNpc Cell Counts and Striatal Optical Density.

Brains from saline and MPTP-treated mice were sectioned (30 μn) and processed for stereological cell counts using the optical fractionator method as described in Cui et al. (PNAS USA 106:8043-8048) Striatal optical densities of TH immunoreactivity were quantified as described in Cui et al.

### In Vivo Microdialysis.

Stereotactic implantation of guide cannula was performed under ketamine/xylazine (65/6 mg/kg i.p.) anesthesia as previously described (Cui et al., PNAS USA 106:8043-8048) using the following striatal coordinates, relative to bregma: anterior-posterior +0.5 mm, lateral −2.0 mm, dorsal-ventral −1.5 mm (from surface of brain). Twenty-four hours after surgery, a microdialysis probe (2-mm membrane. Bioanalytical Systems, Inc.) was inserted into the guide cannula and connected to a low torque-dual channel swivel (Instech Laboratories, Inc., Plymouth Meeting, Pa.), which was connected to a syringe pump perfusing with artificial cerebrospinal fluid (aCSF) at 2 μl/min. After a 2-h equilibration period, dialysates were collected every 15 min for all dopamine release studies. Two baseline fractions were collected, after which the perfusate was switched to aCSF containing 100 mM KCl (with equimolar reduction in NaCl to maintain osmolality) for 15 min to deliver a total of 240 nmoles KCl, followed by a return to normal aCSF for an additional hour. Histological examination subsequent to the experiments was performed to verify the placement of the probe in each animal. Samples from the same animals were measured for the contents of serotonin, DA, and its metabolites. Levels of these molecules and the amount of KCl (delivered to the striatum) were calculated on the basis of the standard curves, probe efficiency (˜8%), flow rate, and duration of sample collection as described in Cui et al. (PNAS USA 106:8043-8048).

### HPLC Measurements of Striatal DA Content.

A 12-channel CoulArray (ESA Inc., Sunnyvale, Calif.) equipped with a highly sensitive amperometric microbore cell (model 5041, ESA Inc., Sunnyvale. CA) was used to analyze the content of DA and its metabolites with the cell potential set at +220 mV. For measurements of total striatal DA content, mice were sacrificed and their striata were dissected out and stored at −80° C. until analysis. On the day of the assay, striatal tissues were sonicated in 50 volumes (wt/vol) of 5% trichloroacetic acid containing 50 ng/ml dihydrobenzylamine as an internal standard. After centrifugation at 15,000×g for 15 minutes at 4° C. the supernatant was removed for HPLC analysis. Briefly, 20 μl samples of dialysates or tissue homogenates were injected manually into a sample injector (with 20 μl sample loop) and eluted on a narrow-bore (ID: 2 mm) reverse-phase C18 column (MD-50, ESA, Inc.) using MD-TM (ESA, Inc.) mobile phase (for striatal homogenates pH was adjusted to 4.25).

### Immuno-Electron Microscopy.

Mice were transcardially perfused with 1% glutaraldehyde/4% paraformaldehyde in 0.1 M sodium cacodylate buffer, pH 7.4. Perfused brains were blocked in the coronal plane and 3 mm slices of striatum (approximately +0.7-4 mm Bregma) were removed, postfixed and then cryoprotected gradually up to 30% sucrose. Tissue was then cut into 50 μm thick coronal sections using a cryostat. Cryostat sections were treated with 1% sodium borohydride in 0.1M TBS for 30 min, washed thoroughly, then blocked in 5% NGS, 1% BSA, 0.1% cold water fish gelatin. 1% glycine, and 1% lysine in 0.1M TBS for 1 hour at room temperature. Tissue was then incubated with polyclonal anti-TH, (1:100, Calbiochem) for 3 nights at 4° C., followed by biotinylated goat anti-rabbit (1:200, Vector Labs) for 2 nights. Sections were then incubated in ExtrAvidin (1:150, Sigma) for one night at 4° C. prior to being reacted with 3,3′-diaminobenzidine (DAB), silver enhanced, gold-toned, and osmicated (1% OsO4). Dehydrated sections were embedded in Spurr epoxy overnight, sectioned (80 nm), stained with uranyl acetate and lead citrate and examined using a Hitachi 7100 electron microscope. A blinded experimenter obtained images at 20,000× of TH-positive terminals, of which a second blinded experimenter quantified the morphology of 50 mitochondria per mouse using ImageJ Version 1.42 (NIH).

### Statistics.

All values are expressed as mean±SEM. Differences between means were analyzed using either 1-way or 2-way ANOVA followed by Newman-Keuls post hoc testing for pairwise comparison using SigmaStat v 3.5 (San Jose, Calif.). For in vivo microdialysis data, areas under the curve were generated using GraphPad Prism v 5.01 (La Jolla, Calif.) followed by a 2-tailed t test. The null hypothesis was rejected when p-value was <0.05.

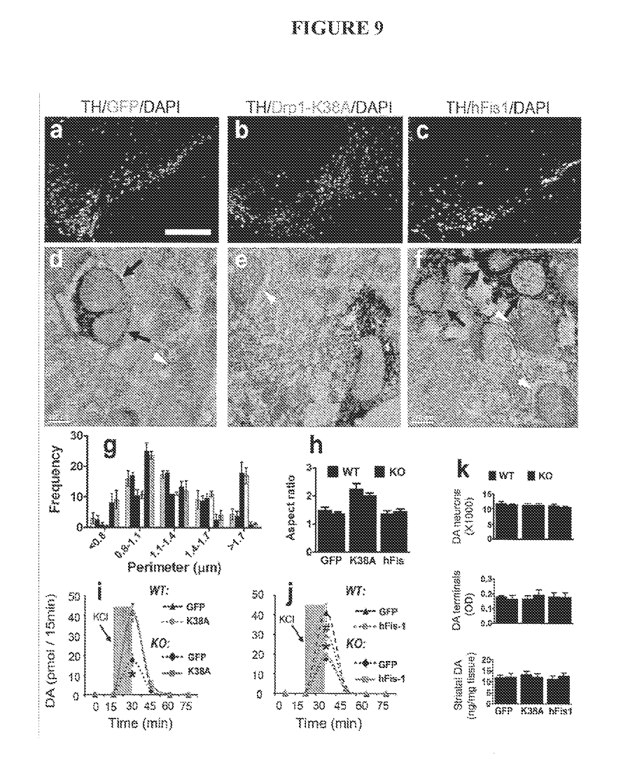

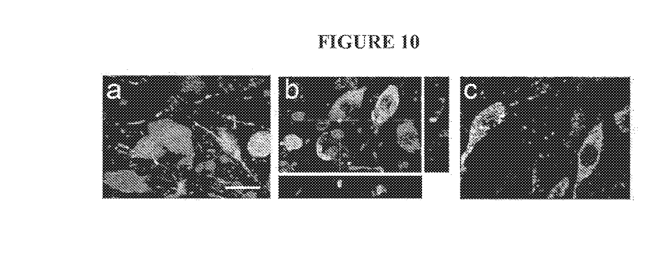

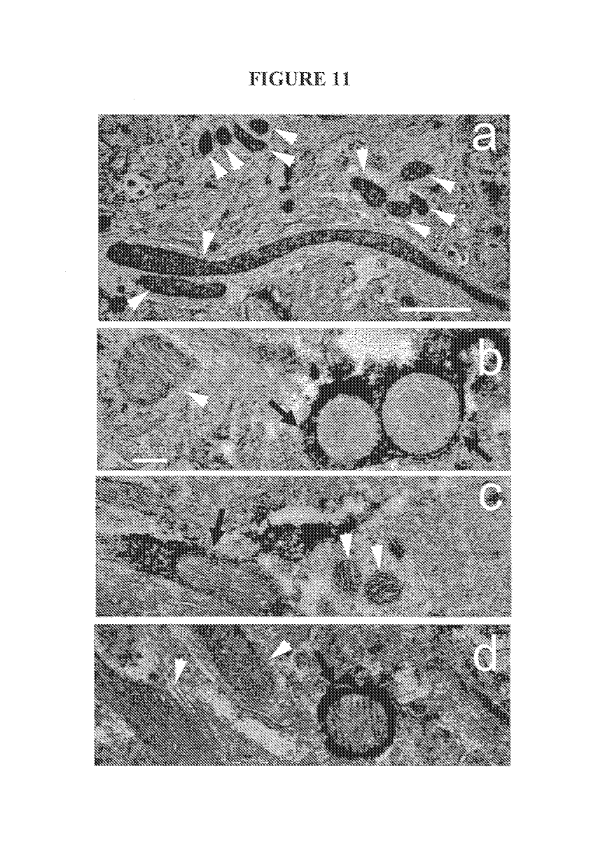

In the present study, Pink 1-null (Pink 1−/−) mice represent a human disease relevant genetic model with age-related impairments in mitochondrial function and evoked nigrostriatal DA release (See Gautier et al. PNAS USA 105, 11364-11369 (2008) and Kitada et al. PNAS USA, 11441-11446 (20076)). The mitochondrial neurotoxin MPTP model provides a model of rather selective nigrostriatal degeneration as seen in PD patients (See Dauer et al. Neuron 39, 889-909 (2003)). Because both pathways of mitochondrial fission and fusion are critical to normal cellular processes and because it is not entirely certain whether promoting fission or fusion is beneficial in PD animal models, both strategies were assessed. First, rAAV2 was injected right above the substantia nigra to deliver Drp1-K38A (a dominant negative mutant of Drp1) to promote fusion, hFis1 to promote fission or enhanced green fluorescent protein (eGFP) as a control. After eight weeks, to allow sufficient time for protein expression, immunofluorescence (FIG. 9, FIG. 10) demonstrated that nigral dopamine (DA) neurons robustly expressed eGFP, Drp1-K38A, or hFis1. Anterograde transport of these proteins to axon terminals in the striatum was also evident. Next, the effects of these proteins on mitochondrial morphology in striatal DA terminals, where mitochondria play a critical role in synaptic release, were determined. Given the heterogeneity of mitochondrial size and morphology in different cell types of this region (FIG. 11), immuno-electron microscopy was performed using tyrosine hydroxylase as a marker for DA structures (FIG. 9d-f). Quantitative morphological measurement of mitochondria in one-year old Pink1−/− and Pink1+/+ littermates confirmed that, as compared to the GFP control group, there was a larger proportion of elongated mitochondria in the group with Drp1-K38A (FIG. 9g,h) and an increased fraction of smaller mitochondria in the hFis 1 group (FIG. 9g). However, hFis1 did not further enhance the roundness of mitochondria in these terminals as indicated by aspect ratio (values approach 1 as the structure becomes more circular). These data also indicate that there was no difference between mouse genotypes regarding the size and shape of mitochondria in DA terminals (FIG. 9g,h, FIG. 11b-d), suggesting mitochondrial dysfunction in mice with germline deletion of Pink 1 and mitochondrial morphology are not necessarily linked.

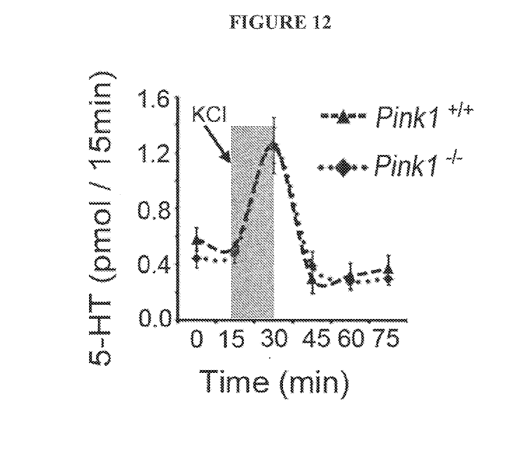

Mitochondria play a crucial role in presynaptic release by providing supports to high-energy demand processes and sequestration of cytosolic Ca²⁺ during normal neurotransmission. In Pink 1-null mice, impairments in evoked nigrostriatal DA release in acute slices have been linked to mitochondrial dysfunction. It was sought to determine whether such impairment also occurred in vivo in freely moving mice and if so, whether promoting fission or fusion would restore this defect. To this end, in vivo microdialysis was used to assess depolarization-induced DA overflow in the striatum via transient perfusion of high-KCl artificial cerebrospinal fluid (aCSF) in ˜12-month old Pink1−/− and wild type littermates. Pink1−/− mice exhibited significantly reduced DA overflow compared to wild type controls (FIG. 9 i,j). Simultaneous quantification of serotonin in these dialysate suggests this deficit was specific to DA (FIG. 12). Impaired DA overflow in Pink1−/− mice was not a result of nigrostriatal damage in these mice (FIG. 9k). Additionally, because the reduced DA overflow in Pink1−/− mice was not due to increased dopamine transporter (DAT) activity, this observation provides in vivo evidence of impaired exocytotic release of DA in these mutant mice. However, after 8 weeks of receiving gene delivery of Drp1-K38A, but not hFis1, a complete restoration of evoked DA overflow was achieved in Pink1−/− mice (FIG. 5i,j). Drp1-K38A did not alter normal synaptic release in Pink1+/+ littermates but hFis1 reduced DA release in these wild type mice (FIG. 9i,j). The changes in DA release observed above occurred in the absence of alterations in total number of nigral DA neurons, striatal DA terminals, or total DA content (FIG. 9k). Together, these results support that, through its well-established mitochondrial fusion-promoting effect, Drp1-K38A is capable of ameliorating the pre-existing DA synaptic dysfunction in Pink1−/− mice.

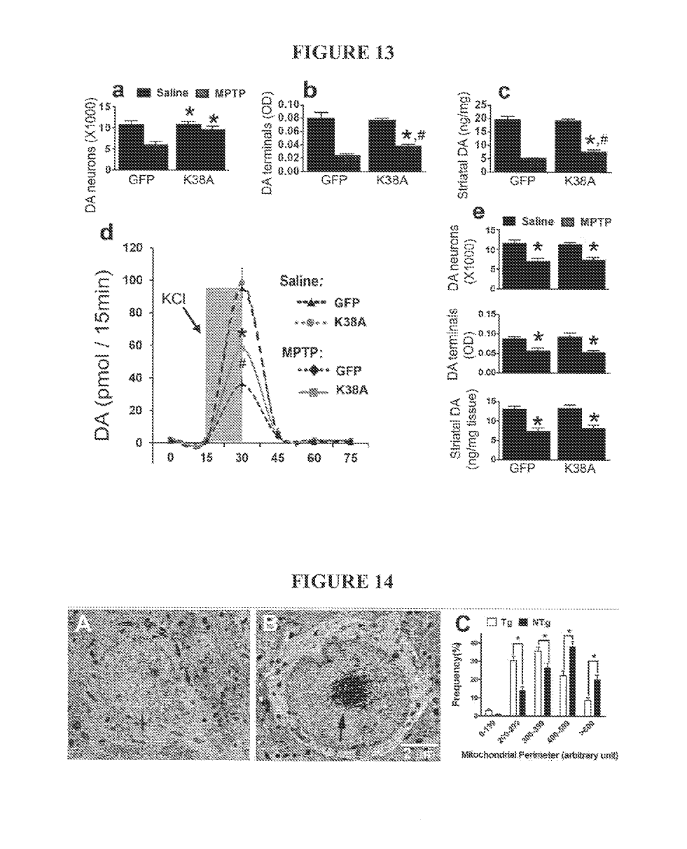

To determine the efficacy of Drp1-K38A in the setting of active neurodegeneration, rAAV2-Drp1-K38A, or rAAV2-GFP was stereotactically delivered to the nigra of C57BL/6 mice. After eight weeks, mice were injected with MPTP daily for 5 days. Drp1-K38A significantly attenuated MPTP-induced degeneration in nigral DA neurons (FIG. 13a), striatal DA terminals (FIG. 13b) and total DA content (FIG. 13c). Considering the substantial amount of nigrostriatal degeneration already present at the time of diagnosis in humans with PD, this scenario was more closely modeled and the neurorestorative potential of blocking Drp1 function was assessed. To this end, mice received MPTP as described above, yet gene therapy was delayed until 7 days after the last injection to allow the lesion to form and stabilize prior to intervention (See Kells et al. J. Neurosci. 30, 9567-9577 (2010)). It was hypothesized that, among the remaining nigrostriatal neurons, there would exist a sizable dysfunctional fraction that could be ameliorated by promoting mitochondrial fusion—a process that could restore mitochondrial function through functional complementation. In mice pretreated with MPTP, Drp1-K38A improved evoked DA overflow (FIG. 13d) despite having no effect on measures of nigrostriatal pathology (FIG. 13e). Together these results demonstrate that promoting mitochondrial fusion by blocking Drp1 function in vivo is neuroprotective against active neurodegeneration and is capable of restoring DA release under pre-existing pathological conditions as seen in human PD.

Provided herein is the first in vivo demonstration that blocking the function of Drp1 is neuroprotective and neurorestorative in mouse models of compromised nigrostriatal pathway. The present in vivo study shows the use of Drp1 as a therapeutic target for PD.

### Example 3

### Huntington's Disease

Huntington's disease (HD) is an autosomal dominant neurodegenerative disorder that is caused by a pathological expansion of CAG repeats within the gene encoding for a 350 kD protein called huntingtin (htt). This polyglutamine expansion within htt is the causative factor in the pathogenesis of HD; however the underlying mechanisms have not been fully elucidated. Nonetheless, it is becoming increasingly clear that mitochondrial dysfunction is likely a key contributor to the pathogenesis of HD. Indeed, indicators of impaired metabolism are evident in presymptomatic HD cases. Pathological alterations in mitochondrial form, function and localization are likely to result in synaptic distinction and neuronal cell death.

### Successful Transduction of Drp1-K38A in R6/2 Mice.

In order to successfully block the effects of the overactive DRP1 protein induced by mutant htt, rAAV2 was used as a means of expressing DRP1-K38A, the dominant negative mutant of DRP1, in the striatum of 3-week old transgenic R6/2 mice and their non-transgenic littermates. R6/2 is a well-characterized HD mouse model in which mitochondrial dysfunction has been demonstrated. It contains approximately 150 CAG repeats and exhibits very rapid and reproducible progression of HD-like symptomology (phenotype, neuropathology and life-span). For instance, these mice begin experiencing motor symptoms and a decline in body weight as early as 5-6 weeks and 10 weeks respectively, and their lifespan is on average 10-13 weeks. Additionally, these mice experience protein aggregation, neuronal dysfunction and decreased striatal and brain size as evidenced by decreased evoked-neurotransmitter release. This latter effect could be mediated by impaired mitochondrial function. DRP1-K38A or GFP control was delivered at 3 weeks to allow sufficient time for gene expression before the onset of motor symptoms at 5 weeks. As shown in FIG. 2. Drp1^{K38A} is expressed in the striatum but is not detectable in the nearby corpus callosum and cortex.

### Mitochondrial Fragmentation in R6/2 Mice.

Because it had not been determined if these mutant mice exhibited mitochondrial fragmentation in the medium striatal neurons, the cell type affected in HD, immunoelectron microscopy was used to measure mitochondrial size and verify whether transgenic R6/2 mice have mitochondrial fragmentation. As shown in FIG. 14, striatal neurons with nuclear huntingtin aggregates in transgenic R6/2 mice have significantly more fragmented mitochondria than non-transgenic mice.

rAAV2-Drp1-K38A Delays Motor Deficits in R6/2 Mice.

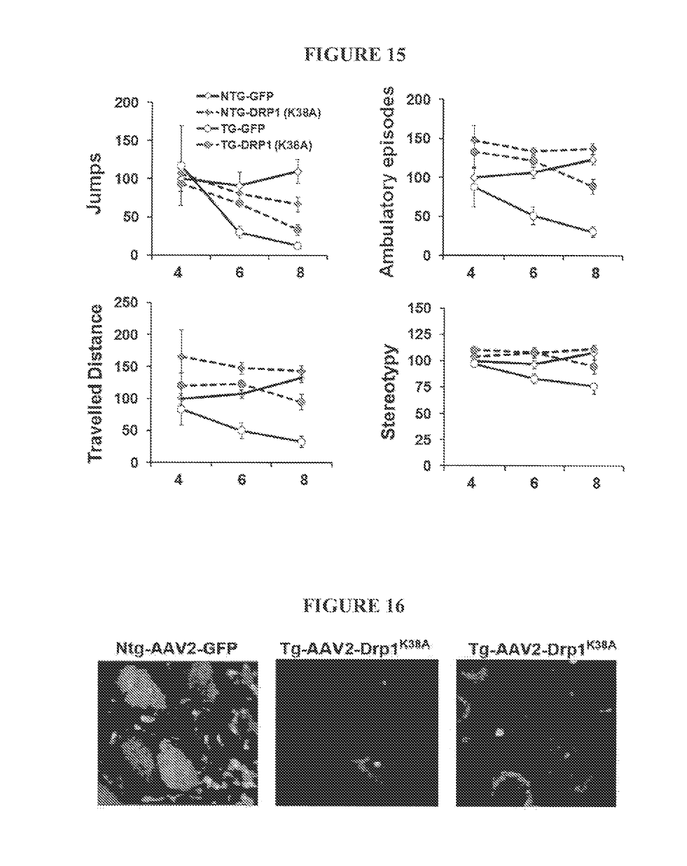

Beginning 1 week after bilateral injection of gene therapy, mice were assessed bi-weekly for their open field locomotion in photobeam chambers up until 8 weeks of age. As seen in FIG. 15, rAAV2-DRP1-K38A attenuated motor deficits in the transgenic R612 mice across all fours measures of locomotion (jumps, travelled distance, ambulatory episodes and stereotypy). Additionally, rAAV2-DRP1-K38A did not appear to adversely affect non-transgenic wild type littermates.

rAAV2-DRP1-K38A Attenuates the Formation of Nuclear Aggregates.

One of the main pathological markers of HD in both human patients and R6/2 mice is the formation of proteolysis-resistant nuclear aggregates by mutated htt. There is much evidence to suggest that in the long run, these protein aggregates, formed from misfolded toxic proteins, confer a toxic effect by interfering with proteasome function, cellular trafficking, autophagic progression and transcription. To determine whether Drp1-K38A had an impact on protein aggregates in striatal medium spiny in the R6/2 mice, immunofluorescence was performed in which striatal sections were co-labeled for both the expression of DRP1-K38A and the presence of nuclear htt aggregates. The confocal microscopy pictures indicated that the expression of DRP1-K38A strikingly attenuated the formation of nuclear aggregates in striatal medium spiny neurons of transgenic animals (FIG. 16).

1. A method of treating a neurological disease or injury in a subject comprising administering to the subject a recombinant adeno-associated virus (rAAV) vector comprising a DRP1 encoding nucleic acid, wherein the DRP1 encoded by the nucleic acid comprises a mutation compared to wild-type DRP1.

2. The method of claim 1, wherein the neurological disease or injury comprises mitochondrial fragmentation, mitochondrial dysfunction or mitochondrial DNA mutation.

3. The method of claim 1, wherein the neurological disease or injury is selected from the group consisting of Parkinson's disease, Alzheimer's disease, Huntington's disease, amyotrophic lateral sclerosis, stroke, and ischemia.

4. The method of claim 3, wherein the neurological disease or injury is Parkinson's disease.

5. The method of claim 1, wherein the vector comprises an AAV compatible plasmid and wherein the plasmid comprises a promoter functionally linked to the DRP1 encoding nucleic acid.

6. The method of claim 5, wherein the plasmid is a pFBGR plasmid.

7. The method of claim 5, wherein the promoter is a cytomegalovirus promoter.

8. The method of claim 1, wherein the vector comprises at least two inverted terminal repeats.

9. The method of claim 1, wherein the DRP1 mutation is K38A.

10. The method of any claim 1, wherein the rAAV is selected from the group consisting of AAV1, AAV2, AAV3, AAV4, AAV5, AAV6, AAV7, AAV8, AAV9, AAV 10 and AAV 11.

11. The method of claim 1, wherein the vector is administered stereotactically into a selected brain region.

12. The method of claim 11, wherein the selected brain region is the substantia nigra.

13. The method of claim 11, wherein the selected brain region is the striatum.

14. The method of claim 11, wherein the selected brain region is the hippocampus.

15. The method of claim 1, wherein the vector is administered intraventricularly.

16. The method of claim 1, wherein the vector is administered by lumbar puncture.
